(19) United States
(12) Patent Application Publication
(10) Pub. No.: US 2019/0076557 A1
(43) Pub. Date: Mar. 14, 2019

(54) HYALURONAN SN-117M COLLOID FOR RADIOSYNOVIORTHESIS AND SYMPTOMATIC THERAPY RELATED APPLICATIONS

(71) Applicant: Serene, LLC, The Woodlands, TX (US)

(72) Inventors: Gilbert R. Gonzales, Tucson, AZ (US); Nigel R. Stevenson, Sugar Hill, GA (US)

(21) Appl. No.: 15/765,559

(22) PCT Filed: Oct. 5, 2016
(86) PCT No.: PCT/US2016/055443

§ 371 Date: Apr. 3, 2018

Related U.S. Application Data
(60) Provisional application No. 62/237,013, filed on Oct. 5, 2015.

(51) Int. Cl. A61K 51/06; A61K 51/12; A61P 19/02; A61K 9/00; A61K 31/728

(52) U.S. Cl. CPC A61K 51/065; A61K 51/1217; A61K 31/728; A61K 9/0019; A61P 19/02

(57) ABSTRACT

A colloidal suspension of hyaluronic acid and Sn-117m is used to treat joint inflammation. In one embodiment, Sn-117m is bonded to hyaluronic acid. Alternately, the Sn-117m can be dissolved in the hyaluronic acid colloidal suspension. This is injected into an inflamed joint, providing short-term and long-term relief.

### RELATED APPLICATIONS

The present application is a national phase application of PCT Application No. PCT/US2016/055443 filed Oct. 5, 2016, which claims priority to U.S. patent application Ser. No. 62/237,013 filed Oct. 5, 2015, the disclosures of which are hereby incorporated herein by reference in their entirety.

### BACKGROUND OF THE INVENTION

A colloid is a mixture of microscopic insoluble particles dispersed or suspended in a second substance, a heterogeneous mixture with properties also intermediate between a solution and a mixture. The particles generally have a diameter between 1 nm and 1000 nm. Many membranes restrict the passage of dispersed colloidal particles more than they restrict the passage of dissolved ions or molecules; i.e. ions or molecules may diffuse through a membrane through which dispersed colloidal particles will not diffuse. The dispersed phase particles are largely affected by the colloidal surface chemistry.

Hyaluronic acid (hyaluronan) is a glycosaminoglycan distributed widely throughout connective, epithelial, and neural tissues. It is one of the chief components of the extracellular matrix, contributes significantly to cell proliferation and migration, and may also be involved in the progression of some malignant tumors. Hyaluronan is a polymer of disaccharides themselves composed of D-glucuronic acid and D-N-acetylglucosamine, linked together via alternating beta-1,4 and beta-1,3 glycosidic bonds. Hyaluronan works in osteoarthritic joints to relieve pain by acting as a cushion and lubricant. Hyaluronan powder produced from human allographic, autographic or synthetic sources may be used as a colloid. Hyaluronic acid forms a colloidal suspension in water or saline.

Radiosynoviorthesis (RSO), also known as radiosynovectomy, is a local intraarticular injection of radionuclides, typically, in colloidal form for radiotherapy and is typically used for the treatment of resistant synovitis of joints after failure of systemic pharmacotherapy, intraarticular steroid injections and other intraarticular therapeutics. RSO is intended to relieve pain and inflammation from rheumatoid arthritis (RA), for which it initially was used, and is accepted as an alternative to surgical synovectomy in cases of inflammatory arthropathies such as osteoarthritis (OA) and hemophiliac arthropathy and is an option for treating chronic synovitis in RA or secondary to inflammatory arthropathies.

### SUMMARY OF THE INVENTION

A radioisotope, specifically Sn-117m combined with a hyaluronic acid colloidal suspension provides a compound which is suitable for intraarticular injection therapy to alleviate symptoms of joint inflammation. Acute and subacute symptom relief is provided by the hyaluronic acid. Long term symptom relief and reversal of inflammatory joint destruction (depending on the underlying joint disorder) is due to the unique therapeutic energy delivered by the isotope Sn-117m and its conversion electron emissions. Further, the hyaluronic acid keeps the Sn-117m within the joint, preventing damage to extra articular tissue.

### DETAILED DESCRIPTION OF THE INVENTION

The present invention provides a compound which is a combination of hyaluronic acid and tin-117m. This compound as a colloidal suspension can then be used in RSO to provide short-term relief and reversal of inflammatory joint destruction.

Joint disorders which can be treated with this composition include rheumatoid arthritis, spondyloarthropathy, psoriatic arthritis, ankylosing spondylitis, reactive arthritis, enteropathic arthritis, other inflammatory joint diseases such as Behçet and lyme disease, calcium pyrophosphate deposition arthritis, pigmented villo-nodular synovitis, hemophilic arthropathy, osteoarthritis, recurrent joint effusion after surgery or prosthesis, other undifferentiated arthritis such as recurrent synovitis, recurrent hydrarthrosis and synovial thickening.

To treat these maladies, the colloidal suspension of hyaluronic acid and Sn-117m is injected into the affected joint. Generally, the dosage of Sn-117m will be from 0.05 μCi to 20 mCi, more preferably in the range of 0.5 mCi to 5 mCi which, of course, will vary depending upon the extent of the disease and the size of the patient and the joint. Likewise, the amount of hyaluronic acid should be an amount effective to provide symptom relief and thus, the ratio of the hyaluronic acid and Sn-117m will vary according to these two requirements.

Although carrier added Sn-117m may be used, no carrier added high specific activity Sn-117m provides many advantages. No-carrier-added Sn-117m can be prepared in an accelerator, such as cyclotron, by transmutation of antimony into no-carrier-added Sn-117m by high-energy proton induced nuclear reactions. No-carrier-added Sn-117m can also be obtained by exposing cadmium 116 to an alpha particle beam as described in U.S. Pat. No. 8,257,681, the disclosure of which is incorporated herein by reference. This permits formation of high specific activity Sn-117m, for example at least 100-1000 or more curies per gram. Current methods provide for 20,000 Ci/g. The tin-117m containing colloidal suspension can be prepared with high specific activity (>1000 Ci/g), medium specific activity (100-1000 Ci/g) or low specific activity (<100 Ci/g) depending on the application required.

The hyaluronic acid, generally in the form of sodium hyaluronate, may be a high molecular weight hyaluronic acid. Generally, the molecular weight should be high enough to maintain the hyaluronic acid within the joint but low enough to provide an injectable fluid. Higher molecular weights provide additional cushioning effect in the joint and thus greater immediate pain relief. The molecular weight should be greater than 1×10⁴ Da, generally at least 1×10⁶ Da, up to to 2×10⁷ Da. Commercially available hyaluronic acids range from 8×10⁵-7×10⁶ Da for ARTZ from Seikagaku Kogyo Company Limited and HYALGAN from Fidia Company Limited, 7×10⁶ Da for SYNVISC® from BioMatrix. The molecular weight of hyaluronic acid in human synovial fluid ranges from 1.6×10⁶ to 10.9×10⁶ Da. Basically the upper limit for the molecular weight is the ability to obtain a fluid colloidal suspension that can be injected into the joint.

Typically the colloidal suspension of hyaluronic acid will be in an aqueous carrier, such as a saline solution. The concentration of the hyaluronic acid can vary from 0.01 to 3% (W/V) typically 0.3 to 1%. Again the upper limit of the concentration depends on the ability to inject the colloidal suspension into the joint.

The Sn-117m can be blended with the hyaluronic acid or can be chemically bonded to the hyaluronic acid. The colloidal suspension of hyaluronic acid can be mixed with Sn-117m colloidal compounds (different than hyaluronic acid), such as those disclosed in WO2013/096776, the disclosure of which is hereby incorporated herein by reference. In this embodiment, a colloidal suspension of hyaluronic acid is mixed with a Sn-117m containing non-hyaluronic acid colloidal suspension to produce a homogeneous mixture without phase separation. The combination will include sufficient Sn-117m to provide the desired dosage.

Alternately, a colloidal suspension of hyaluronic acid can be mixed with a Sn-117m solution thereby coating/impregnating and fixing the hyaluronic acid particulates with Sn-117m. The Sn-117m is usually obtained as a water soluble compound, such as a nitrate and is dissolved in the hyaluronic acid colloidal suspension. Again, sufficient Sn-117m is added to provide the desired radiation dosage.

Hyaluronic acid can be chemically bonded to Sn-117m. This may be achieved by attaching the tin-117m atoms at the amino acid terminals, OH terminals or other suitable locations on the hyaluronan chains.

One particular way of bonding tin-117m to the hyaluronic acid is to use a tether such as an amino terminated tether attached to hydroxy groups of the hyaluronan. This process is disclosed in U.S. Pat. No. 6,409,990, the disclosure of which is incorporated herein by reference. The disclosed process activates the hydroxyl group with bromine followed by a reaction with amino ethane thiol to produce the amine terminated tether. The amine can then be reacted with a tin-containing compounds such as tin-117m aminobenzyl-DOTA to form a tin-117m containing hyaluronic acid compound.

The following experiments demonstrate mixing of SnCl₄ with hyaluronic acid resulting in colloidal suspension. For these experiments, stable tin was used.

### Experiment 1: Hyaluronic Acid Solution added to SnCl₂ Particles:

0.1 g of hyaluronic acid dissolved in DI water to get 1.01 g of solution
Mixed 284 uL of 1M urea+285 uL of 0.02M Sn₂Cl+30 uL of 4M HCl
   Heat at 90-95° C. for four hours
   Colloidal suspension formed
   Yellow precipitate and solution
200 uL of 1% hyaluronic acid solution added to colloidal suspension. Gelatinous-like consistence of product with some precipitation
Similar result for 0.5% hyaluronic acid solution

### Experiment 2: SnCl₄ Particles in Hyaluronic Acid Solution:

Mix 285 uL of 1M urea +285 uL of 0.02M SnCl₄.5H₂O+30 uL of 4M HCl
Produce colloid by heating at 90° C. for four hours
Add hyaluronic acid solution
Light-hazy precipitate

The radiation dosage injected into a joint can range from 0.025 to 20 mCi. The radiation dose typically used for RSO is between 0.5 mCi and up to 6 mCi in human medium and large joints and between 0.25 and 3 mCi in human small joints. To provide a hormetic effect, the dosage can be 1/10^{th} to 1/100^{th} of these typical dosages, such as 0.05 to 0.6 mCi or 0.005 to 0.06 mCi for larger joints and 0.025 to 0.3 mci or 0.0025 to 0.03 mCi for small joints. These dosages are for humans. Animal dosages will vary based on size. The Sn-117m colloidal suspension in any of the above examples can be utilized in animal species that include humans, dogs, horses, cats and others.

The tin and hyaluronic acid combination is used to treat inflamed joints by injecting an effective concentration of the hyaluronic acid tin-117m combination. Generally about 0.1 mL per kilogram of body weight of the hyaluronic acid solution is injected into the joint. More or less can be injected in order to establish the effective radiation dose. Typically, with synovectomy procedures for humans, about 2 mL of a 1% (W/V) hyaluronic acid is used. Again, this can vary based on the size of the joint. Prior to injection, it may be desirable to remove a portion of the synovial fluid from the joint. The injection can be repeated generally at one-week intervals.

Thus the present invention alleviates the symptoms of joint inflammation and provides long-term relief and reversal of inflammation or joint destruction due to the therapeutic energy delivered by tin-117m. The limited effective distance of the conversion electron emitted by the tin-117m ensures that only nearby tissue is in any way affected by the conversion electron. Sn-117m does not emit β radiation. No tissue greater than or more than about 300 μ from the tin-117m will be affected by the emitted radiation. Further, the hyaluronan prevents the Sn-117m from passing through the joint membranes. This prevents extra articular tissues from being exposed to radiation. Thus the present invention treats joint inflammation without damaging nearby healthy tissue or extra articular tissue.

This has been a description of the present invention, along with the preferred method of practice, however, the invention itself should only be defined by the appended claims.

What is claimed is:

1. A composition comprising the colloidal suspension of hyaluronic acid and Sn-117m.

2. The composition claimed in claim 1 wherein said Sn-117m is bonded to a colloid other than hyaluronic acid.

3. The composition claimed in claim 1 wherein said Sn-117m is bonded to said hyaluronic acid.

4. The composition claimed in claim 1 wherein said Sn-117m is blended with a hyaluronic acid colloidal suspension .

5. The composition claimed in claim 1 wherein said Sn-117m is bonded to said hyaluronic acid via a tether.

6. The composition claimed in claim 5 wherein said Sn-117m is bonded to aminobenzyl-DOTA, in turn bonded to said tether.

7. A method of treating a disorder selected from the group consisting of rheumatoid arthritis, spondyloarthropathy, psoriatic arthritis, ankylosing spondylitis, reactive arthritis, enteropathic arthritis, other inflammatory joint diseases such as Behçet and lyme disease, calcium pyrophosphate deposition arthritis, pigmented villo-nodular synovitis, hemophilic arthropathy, osteoarthritis, recurrent joint effusion after surgery or prosthesis, other undifferentiated arthritis such as recurrent synovitis, recurrent hydrarthrosis and synovial thickening comprising administering a dose of the composition of claim 1 into the affected joint in an amount effective to treat the disorder.

8. The method claimed in claim 7 wherein the dose is a hormetic dose injected into said join wherein said hormetic dose is from 0.025 to 0.06 mCi.

9. The method claimed in claim 7 wherein said dose is 0.05 mCi to 20.0 mCi.

10. The method claimed in claim 7 wherein said Sn-117m is no-carrier-added tin-117m having an activity of at least 1000 Ci/g.
